(19) United States
(12) Patent Application Publication
(10) Pub. No.: US 2008/0305053 A1
(43) Pub. Date: Dec. 11, 2008

(54) Fluoride-releasing strips for tooth

(75) Inventors: Bor-Shiunn Lee, Taipei (TW); Da-Ming Wang, Taipei City (TW); Juin-Yih Lai, Taoyuan County (TW); Chun-Pin Lin, Taipei (TW); Chun-Yu Chen, Taipei City (TW); Fang-Yu Liu, Taipei City (TW)

Correspondence Address: WPAT, PC, 7225 BEVERLY ST., ANNANDALE, VA, 22003, US

(73) Assignee: NATIONAL TAIWAN UNIVERSITY, Taipei City (TW)

(21) Appl. No.: 11/760,894

(22) Filed: Jun. 11, 2007

(51) Int. Cl. A61K 8/21; A61Q 11/00

(52) U.S. Cl. 424/52

(57) ABSTRACT

The present invention discloses fluoride-releasing strips for tooth, wherein the fluoride-releasing strips comprises a fluoride-containing solution for releasing fluorine ion and a support substrate. A formula of fluoride-containing solution comprises a fluoride solution, at least one buffer, at least one moisturizer and a tackiness agent. The support substrate is waterproof material and a fluoride-containing solution is applied on the support substrate.

### BACKGROUND OF THE INVENTION

1. Field of the Invention

The present invention is generally related to a strip, and more particularly to a fluoride-releasing strip for tooth.

2. Description of the Prior Art

Oral healthiness is important to the state of human health. In addition, chewing malfunction will result in insufficient absorption of nutrients and thereby affect the healthiness of the whole body. Among a variety of reasons affecting oral healthiness, dental caries is accounted for an important one. For example, dental restoration, endodontic treatment, prosthetic tooth fabrication, and tooth extraction almost all result from dental caries. Therefore, most of the medical treatment resources for maintaining oral healthiness are used to cure the follow-up problems of dental caries. Although commercially available fluoride-containing products for dental caries prevention are numerous, the situation of dental caries for school children becomes serious in Taiwan. According to the report, permanent tooth dental caries rate for twelve-year-old children is about 89˜94% and it is about 4.5˜5.5 dental caries per person in average, much more than that in well-developed countries, such as European or American countries. Therefore, academic organizations figure out that the current environmental factors will accelerate the possibility of having dental caries for school children if no effective action is taken. According to the classification by the world health organization, the situation of dental caries for school children in Taiwan will change from a serious state to a very serious state.

In light of the above mentioned matter, in order to prevent dental caries, the effectiveness of these fluoride-containing products and the safety and convenience in the use of these fluoride-containing products are important factors, besides having good personal tooth cleaning habit. Providing a simple fluoride-releasing strip for tooth becomes an important research subject.

### SUMMARY OF THE INVENTION

In light of the above background, in order to fulfill the requirements of the industry, the invention provides a fluoride-releasing strip for tooth to prevent dental caries.

One object of the present invention is to provide a fluoride-releasing strip for tooth. The fluoride-releasing strip for tooth comprises a fluoride-containing solution for releasing fluorine ions and a support substrate. The fluoride-containing solution is applied on the support substrate.

### BRIEF DESCRIPTION OF THE DRAWINGS

FIG. 1 shows the relationship of the fluoride concentration on the enamel surface and fluoride-containing strip tack time (n=15);

FIG. 2 shows the result of demineralization inhibition paired-t test for 2 weeks [*: there is remarkable difference, compared to the control group. (n=10)(p<0.05)];

FIG. 3 shows the result of demineralization-inhibition paired-t test for 4 weeks [*: there is remarkable difference, compared to the control group. (n=5)(p<0.05)];

FIG. 4 shows the result of remineralization-promotion paired-t test for 3 days [*: there is remarkable difference, compared to the control group. (n=10)(p<0.05)];

FIG. 5 shows the result of remineralization-promotion paired-t test for 1 week [*: there is remarkable difference, compared to the control group. (n=10)(p<0.05)]; and

FIG. 6 shows the result of remineralization-promotion paired-t test for 2 weeks [*: there is remarkable difference, compared to the control group. (n=10)(p<0.05)].

### DESCRIPTION OF THE PREFERRED EMBODIMENTS

What is probed into the invention is a fluoride-releasing strip for tooth. Detail descriptions of the processes and composition structures will be provided in the following in order to make the invention thoroughly understood. For those who are skilled in the art, generally the weight ratio is used to represent the ratio of each composition in the formula. Therefore, in the following description of the invention, the weight ratio is used to represent the ratio of each composition in the formula. Obviously, the application of the invention is not confined to specific details familiar to those who are skilled in the art. On the other hand, the compositions and processes that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, a fluoride-releasing strip for tooth is provided. The fluoride-releasing strip for tooth comprises a fluoride-containing solution for releasing fluorine ions and a support substrate. The fluoride-containing solution is applied on the support substrate. When using the fluoride-containing strip, one hand contacts the support substrate directly in order to handle the fluoride-containing strip and has the fluoride-containing surface face and contacted with teeth. It is very convenient for use. One formula for the fluoride-containing solution comprises a fluoride solution, at least one buffer, at least one moisturizer for inhibiting water dissipation, and a tackiness agent. The fluoride-ion concentration in the fluoride solution is about more than or equal to 1000 ppm. When the fluoride ions are from sodium fluoride solution, the fluoride-ion concentration is about 0.2 gNaF/100 mlH₂O. In this embodiment, the support substrate is waterproof material. Preferably, the support substrate comprises one substance selected from the group consisting of the following: handi-wrap, waterproof non-woven fabrics, and plastic films.

The buffer is used to adjust the pH value of the fluoride-containing solution. In the invention, adjusting the pH value directly by the addition of alkaline substance has the many disadvantages, such as causing local viscosity to rise drastically and producing precipitation. When the pH value is raised up to 4, condensation occurs. Therefore, the invention uses the buffer to adjust pH value. The viscosity of the mixture is thus increased gradually. The original pH value of the mixture is about 2.5˜3.5. The pH value of the fluoride-containing solution can be adjusted to become weak acidic or neutral. In this embodiment, the preferred pH value of the fluoride-containing solution has two ranges. The first range is 6.2˜6.5 and the pH value of the fluoride-containing solution is neutral. The second range is about 4˜5. When the pH value of the fluoride-containing solution is adjusted to around 4˜5, teeth may be slightly eroded to have micropores on the surface thereof so as to have fluorides enter the micropores and to thereby release fluoride ion for a longer period of time. Thus, the dental caries prevention can be promoted. In addition, the composition of the buffer comprises one compound selected from the group consisting of the following or any combination thereof: phosphates, nitrates, acetates, nitrites, borates, and oxalates.

In this embodiment, the composition of the moisturizer comprises one compound selected from the group consisting of the following or any combination thereof: glycerin, butylene glycol, hyaluronic acid, ceramides, hyaluronic acid, and collagen. But, it is not limited to the above examples. A preferred example according to the invention, the moisturizer is glycerin with concentration of 5.0 g/100 mlH₂O.

The preferred material for the tackiness agent is polyacrylic acid, such as a commercial product called carbopol. According to the invention, the preferred concentration for carbopol is between 2.5 g/100 mlH₂O and 5.0 g/100 mlH₂O. More preferably is 3 g/100 mlH₂O. When the concentration is more than 4 g carbopol/100 mlH₂O, the efficiency of the mass transfer of fluoride substance is reduced.

In another embodiment of the present invention, a method for forming the fluoride-containing solution is disclosed. At first, a fluoride solution is provided, where the fluoride-ion concentration of the fluoride solution is about more than or equal to 1000 ppm. At least one buffer is then added to the fluoride solution to form a first mixture solution. A moisturizer is then mixed with the first mixture solution to form a second mixture solution. Finally, a tackiness agent is added to the second mixture solution and then the second mixture solution is blended to form a colloidal fluoride-containing solution.

In another embodiment of the present invention, a method for forming the fluoride-containing solution is disclosed. At first, a sodium fluoride (NaF) solution is provided where the fluoride-ion concentration of the sodium fluoride (NaF) solution is about more than or equal to 1000 ppm. Next, at least one buffer is added to the fluoride solution to form a first mixture solution where the buffer comprises KH₂PO₄ and K₂HPO₄ with a molar ratio of 2:3. A moisturizer, such as glycerin, is then added and mixed with the first mixture solution to form a second mixture solution. The concentration of the moisturizer is about 5.0 g/100 mlH₂O (relative to the concentration of the final fluoride-containing solution). A tackiness agent, such as polyacrylic acid, is added to the second mixture solution to form a third mixture solution. The concentration of the tackiness agent is about 3.0 g/100 mlH₂O (relative to the concentration of the final fluoride-containing solution). Then, the third mixture solution is blended for about 6˜8 hrs until the third mixture solution becomes colloidal to form the fluoride-containing solution.

### EXAMPLE 1

The experiment uses a molar tooth extracted from human being in a dental clinic. At first, the collected tooth is pretreated by a supersonic tooth-cleaning machine in order to remove dental calculus and stain on the surface thereof. Next, a low speed saw is used to cut the side surface portion of the tooth crown (thickness of about 12 mm). A high speed dental drill is used to grind the tooth. Several pieces of squared specimens with a side length of 0.5 cm are obtained. Finally, the specimens are placed in a supersonic tank to be cleaned for future use.

NaF is dissolved in deionized water and thus a 4000 ppm NaF solution is prepared. KH₂PO₄ and K₂HPO₄ with a molar ratio of 2:3 are added as the buffer composition and 5.0 g/100 mlH₂O of glycerin is added to inhibit water dissipation. The solution is then stirred until uniform and 3.0 g/100 mlH₂O of carbopol is added. The solution is stirred for 6˜8 hrs at a room temperature to become colloidal.

Waterproof handi-wrap is used as the support substrate for the strip. The preparation method of a fluoride-releasing strip for tooth is described in the following. The handi-wrap used as the support substrate is wrapped on a glass plate that is laminated with a release film. The prepared colloidal solution is coated on the handi-wrap by an adjustable blade to form a 500 μm film, according to the general thickness 200˜600 μm for a scraped film. Several blocks with a pre-measured area are cut by scissors and weighted by an electronic balance. Thus, the required quantity of the solution per unit area for forming the strip with an average thickness of 500 μm is calculated. Therefore, in the experimental operation, according to the surface area of the tooth specimen, the fluoride-containing solution is weighted and applied on the handi-wrap and then coated on the enamel surface of the tooth specimen.

Each tooth is divided into four pieces. The fluoride-containing solution is coated on the enamel surface of the tooth specimen, on which the strip is attached at 35° C. for 0 hr (control), 4 hrs, 8 hrs, and 24 hrs. The elemental analysis of the surface is done by scanning electron microscope (SEM)/energy dispersive X-ray spectrometer. The energy dispersive X-ray spectrometer utilizes a high energy electron beam to bombard the to-be-tested surface to have the inner-shell electrons of the element on the surface ionized and to have thereby the outer-shell electron jump in to an inner shell vacancy to release X-ray and then uses an X-ray detector to detect the X-ray emitted from the element on the bombarded surface so as to determine element species and the relative ratio between elements according to the energy intensity of the detected X-ray. Therefore, crack or damage and uneven curved surface are tended to result in the errors on the measurement and analysis. Thus, each specimen has been enlarged 100 times to have five perfect blocks on the surface thereof for elemental analysis so as to prevent the errors on data due to the surface imperfection while using low magnification (×40). Data are selected according to the relative ratio between Ca and P in the elemental analysis. The major composition of tooth enamel is Ca₁₀(PO₄)₆(OH)₂ and thus the theoretical relative ratio between Ca and P (Ca/P ratio) is 1.67. Although the Ca/P ratio may be different for different specimens and may have some variation due to measurement errors, generally Ca/P ratio=1.5˜2.0 is used as the approximate standard. Therefore, if there is a large error in the Ca/P ratio due to the defect on the surface of the specimen, the corresponding data will be deleted.

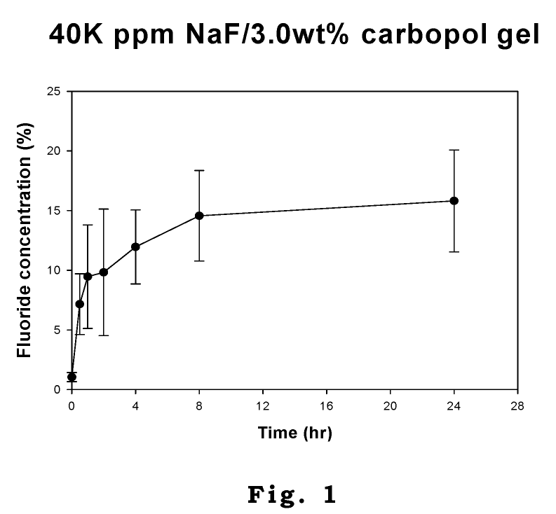

As shown in FIG. 1, the fluoride concentration of the tooth enamel surface is saturated after the strip is attached for 8 hrs, this is, the fluoride concentration of the tooth surface can reach the maximum value for 8 hrs of strip attaching. According the report, in the case of average bed time of 8 hrs and 40 minutes, utilizing the strip according to the invention during bed time can make the strip have the best fluoride releasing effect and additionally can prevent bad appearance while using in day time and strip ablation while eating.

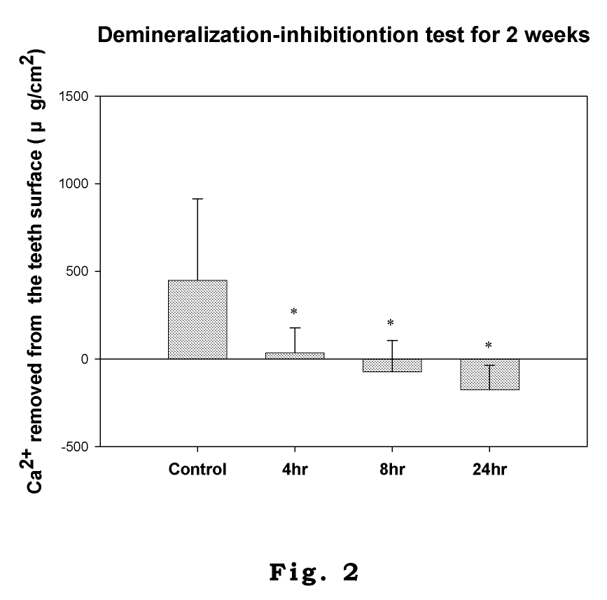

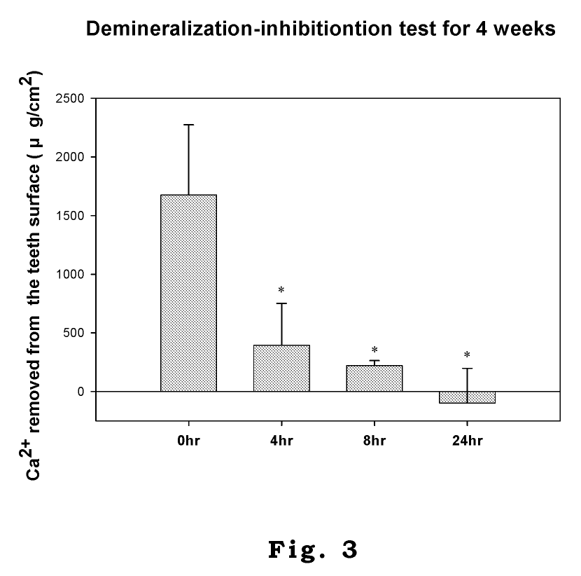

After applied high-concentrated fluoride, the enamel surface of the tooth has calcium fluoride formed thereon which is an important medium for dental caries resistance. Fluoride-ions are released because of gradual dissolution of CaF₂ to have higher fluoride-ion concentration around the tooth surface so as to continuously inhibit tooth demineralization and promote remineralization. Therefore, after using fluoride compound, the precipitation capability and quantity of CaF₂ on the tooth surface are directly related to the acid resistance of the tooth. However, calcium fluoride precipitated on the sound enamel surface is easy to fall off by tiny physical force due to lack of chemical bonding with the tooth surface. On the contrary, if the tooth surface is eroded by acids to have micropores partially, calcium fluoride can precipitate on a larger surface area and the calcium fluorides formed in pores are not easily disturbed by external force to result in falling off. Therefore, better acid resistance can be obtained. Thus, in the experiments of 2 weeks and 4 weeks, because adhering strips and soaking in acidic solution are performed repeatedly, in the process of soaking demineralization solution, the tooth specimen possibly has micropores due to slight erosion on the surface of the tooth specimen and then in the following process of adhering strips, fluoride ions enter these micropores, and these fluoride ions together with calcium ions in the solution form calcium fluoride precipitation while soaking in acidic solution. Thus, after repeatedly operations for 2 weeks and 4 weeks, the capability of inhibiting demineralization for experimental groups is obviously promoted, as shown in FIGS. 2 and 3.

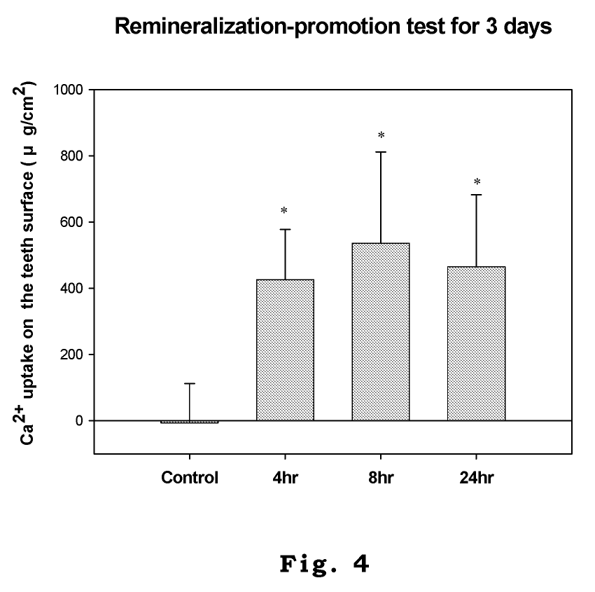

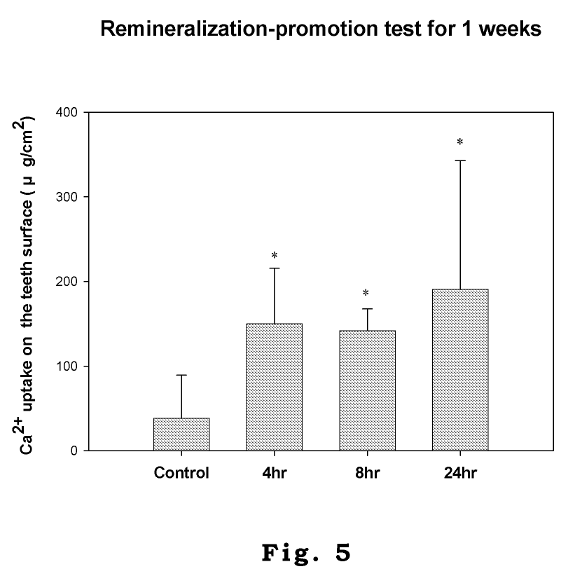

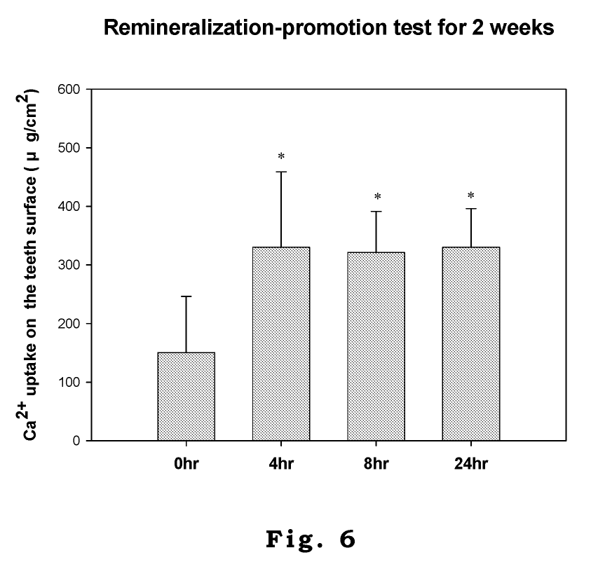

Referring to FIGS. 4, 5, and 6, after the experiments for 3 days, 1 week, 2 weeks, regardless of the period of time of adhering strips and the number of times in adhering strips, the effect of remineralization is obviously promoted. During the process of soaking the tooth specimen in acidic solution, the initial-stage dental caries on the tooth specimen occurs so that the tooth surface has micropores formed on the tooth specimen. Fluoride ions easily adsorb in the micropores through diffusion and then these fluoride ions together with calcium ions in the solution form calcium fluoride precipitation while soaking in remineralization solution. Therefore, regardless of adhering strips for 4 hrs, 8 hrs, or 24 hrs, the effect of remineralization is obviously promoted.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method for forming a colloidal fluoride-containing solution being used in a fluoride-releasing strip for tooth, comprising:
providing a fluoride solution;
adding at least one buffer to said fluoride solution to form a first mixture solution;
mixing a moisturizer with said first mixture solution to form a second mixture solution; and
adding a polyacrylic acid based tackiness agent to said second mixture solution and then blending to form said colloidal fluoride-containing solution, and said buffer is used to adjust the pH value of the fluoride-containing solution without causing local viscosity rising drastically and forming precipitation.

18. The method according to claim 17, wherein said fluoride solution is sodium fluoride (NaF) solution.

19. The method according to claim 17, wherein the fluoride-ion concentration in said fluoride solution is more than or equal to 1000 ppm.

20. The method according to claim 17, wherein the concentration of polyacrylic acid in said fluoride-containing solution is from 2.5 g/100 ml H₂O to 4 g/100 ml H₂O.

21. The method according to claim 17, wherein the pH value of said fluoride-containing solution is more than or equal to pH 4.

22. The method according to claim 17, wherein the pH value of said fluoride-containing solution is about 4˜5.

23. The method according to claim 17, wherein the pH value of said fluoride-containing solution is about 6.2˜6.5.

24. The method according to claim 17, wherein the composition of said buffer comprises one compound selected from the group consisting of the following or any combination thereof: phosphates, nitrates, acetates, nitrites, borates, and oxalates.
